(19) United States
(12) Patent Application Publication
(10) Pub. No.: US 2007/0142378 A1
(43) Pub. Date: Jun. 21, 2007

(54) Method of treating restless legs syndrome and/or periodic limb movement disorder with (2s,3s) 2-(3-chlorophenyl)-3,5,5-trimethyl-2-morpholinol

(75) Inventor: Jean-Christophe Barland, Middlesex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Smith Kline Beecham Corporation

(21) Appl. No.: 10/595,885

(22) PCT Filed: Nov. 19, 2004
(86) PCT No.: PCT/EP04/13248

§ 371 Date: May 18, 2006

(30) Foreign Application Priority Data
Nov. 21, 2003 (GB) 0327195.4

(51) Int. Cl. A61K 31/5375

(52) U.S. Cl. 514/237.8

(57) ABSTRACT

Methods for treating Restless Legs Syndrome and/or Periodic Limb Movement Disorder (PLMD) using (+)-(2S,3S)-2-(3-chlorophenyl)-3,5,5-trimethyl-2-morpholinol or a salt or solvate thereof are disclosed.

This invention relates to a novel use of (+)-(2S,3S)-2-(3-chlorophenyl)-3,5,5-trimethyl-2-morpholinol, in particular its use in the treatment of Restless Legs Syndrome, or its use in the treatment of Periodic Limb Movement Disorder (PLMD).

### BACKGROUND OF THE INVENTION

Bupropion hydrochloride, (±)-1-(3-chlorophenyl)-2-[(1,1 -dimethylethyl)-amino]-1-propanone hydrochloride, is the active ingredient of Wellbutrin® which is marketed in the United States for the treatment-of depression. It is also the active ingredient of Zyban® which is marketed in the United States as an aid to smoking cessation. Bupropion is an inhibitor of the neuronal uptake of noradrenaline (NA), and dopamine (DA), does not inhibit monoamine oxidase and has a negligible effect on the neuronal uptake of seretonin. While the mechanism of action of bupropion, as with other antidepressants, is not fully understood, it is presumed that this action is mediated by noradrenergic and/or dopaminergic mechanisms. Initial clinical evidence suggested Wellbutrin® to be a selective inhibitor of noradrenaline (NA) at doses that are predictive of antidepressant activity in animal models (Ascher, J. A., et al., Journal of Clinical Psychiatry, 56: p. 395-401,1995). A more recent analysis (Stahl, S. M. et al., Prim. Care Companion. Journal of Clinical Psychiatry, 6(4), p 159-166, 2004) concludes that bupropion acts via dual inhibition of norepinephrine and dopamine reuptake, having slightly greater functional potency at the dopamine transporter.

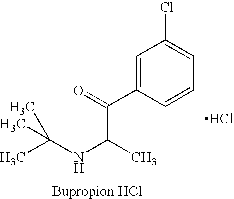

Bupropion is extensively metabolized in man as well as laboratory animals. Urinary and plasma metabolites include biotransformation products formed via hydroxylation of the tert-butyl group and/or reduction of the carbonyl group of bupropion. Four basic metabolites have been identified. They are the erythro- and threo-amino alcohols of bupropion, the erythro-amino diol of bupropion (found in urine but not in plasma), and a morpholinol metabolite.

The morpholinol metabolite (±)-(2R*,3R*-2-(3-chlorophenyl)-3,5,5-timethyl-2-morpholinol is believed to be formed from hydroxylation of the tert-butyl group of bupropion.

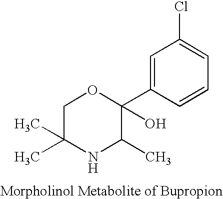

It was discovered that despite the (−) form of the morpholinol metabolite predominating significantly in human plasma samples, it was the (+) enantiomer, (+)-(2S,3S)-2-(3-chlorophenyl)-3,5,5-trimethyl-2-morpholinol in which the optimal monoamine reuptake inhibitory activity resides (WO 99/37305), hereinafter referred to as the compound of formula (I):

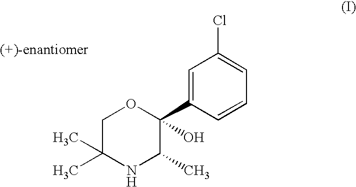

The compound of formula (I) and its salts and solvates have been disclosed as being of use in the treatment of depression (including major depressive disorder (MDD), bipolar depression (type I and II), major (unipolar) depression and depression with atypical features (eg. lethargy, over-eating/obesity, hypersomnia)), attention deficit hyperactivity disorder (ADHD), obesity, migraine, pain (including neuropathic pain, eg. diabetic neuropathy, sciatica, non-specific lower back pain, multiple sclerosis pain, fibromyalgia, HIV-related neuropathy, neuralgia such as as post-herpetic neuralgia and trigeminal neuralgia and pain resulting from physical trauma, amputation, cancer, toxins or chronic inflammatory conditions), sexual dysfunction (including inhibited sexual desire (low libido), inhibited sexual arousal or excitement, orgasm dysfunction, inhibited female orgasm, inhibited male orgasm, hypoactive sexual desire disorder (HSDD), female sexual desire disorder (FSDD) and sexual dysfunction side-effects induced by treatment with antidepressants of the SSRI-class), Parkinson's disease (including relief from the symptoms of Parkinson's disease which include, but are not limited to, locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), Alzheimer's disease, or addiction to cocaine or nicotine-containing (especially tobacco) products (WO 99/37305 and US2003-0064988; both Glaxo Group Limited).

US2003-0032643 (Glaxo Group Limited) discloses the use of the compound of formula (I) and its salts and solvates in the treatment of seasonal affective disorder, chronic fatigue, narcolepsy and cognitive impairment.

US2003-0083330 (Glaxo Group Limited) discloses the use of the compound of formula (I) and its salts and solvates in the treatment of addiction to alcohol.

WO 00/51546 and WO 01/62257 (both Separacor Inc) disclose the use of a bupropion metabolite in the treatment of a disorder that is ameliorated by the inhibition of neuronal monoamine reuptake, sexual dysfunction (including erectile dysfunction), an affective disorder (including depression, anxiety disorders, attention deficit hyperactivity disorder, bipolar and manic conditions, sexual dysfunction, psycho-sexual dysfunction, bulimia, obesity or weight gain, narcolepsy, chronic fatigue syndrome, seasonal affective disorder, premenstrual syndrome, and substance addiction or abuse), nicotine addicton, a cerebral function disorder (including senile dementia, Alzheimer's type dementia, memory loss, amnesia/amnestic syndrome, epilepsy, disturbances or consciousness, coma, lowering of attention, speech disorders, Parkinson's disease, Lennox syndrome, autistic disorder, autism, hyperkinetic syndrome, schizophrenia, cerebral infarction, cerebral bleeding, cerebral auteriosclerosis, cerebral venous thrombosis and head injury), epilepsy, smoking cessation and incontinence.

Dopaminergic agents such as L-Dopa, pergolide and agonists at the D₃ subtype of the D₂ receptor such as ropinerole and pramipexole are recommended by physicians as first choice treatments in RLS (Stiasny K., et al., Restless legs syndrome and its treatment by dopamine agonists, Parkinsonism and Related Disorders, 7, p21-25, 2001; Chesson A. L., Practice parameters for the treatment of restless legs syndrome and periodic limb movement disorders, Sleep, 22(7), p 961-968, 1999). Reference is also made to a small retrospective study of bupropion SR in depressed patients (Nofzinger E. A. et al., Bupropion SR reduces periodic limb movements associated with arousals from sleep in depressed patients with periodic limb movement disorder, Journal of Clinical Psychiatry, 61, p858-862, 2000).

### SUMMARY OF THE INVENTION

The present invention provides the use of (+)-(2S,3S)-2-(3-chlorophenyl)-3,5,5-trimethyl-2-morpholinol or a salt or solvate thereof or pharmaceutical compositions thereof in the manufacture of a medicament for the treatment of Restless Legs Syndrome (RLS).

A further aspect of the invention provides a method of treating Restless Legs Syndrome (RLS) in a mammal (human or animal subject) comprising the administration to said subject of an effective amount of (+)(2S,3S)-2-(3-chlorophenyl)-3,5,5-trimethyl-2-morpholinol or a salt or solvate thereof or pharmaceutical compositions thereof.

One further aspect of the present invention provides the use of enantiomerically pure (+)-(2S,3S)-2-(3-chlorophenyl)-3,5,5-trimethyl-2-morpholinol or a salt or solvate thereof or pharmaceutical compositions thereof in the manufacture of a medicament for the treatment of Restless Legs Syndrome (RLS).

A yet further aspect of the invention provides a method of treating Restless Legs Syndrome (RLS) in a mammal (human or animal subject) comprising the administration to said subject of an effective amount of enantiomerically pure (+)-(2S,3S)-2-(3-chlorophenyl)-3,5,5-timethyl-2-morpholinol or a salt or solvate thereof or pharmaceutical compositions thereof.

A further aspect of the present invention provides the use of (+)-(2S,3S)-2-(3-chlorophenyl)-3,5,5-trimethyl-2-morpholinol or a salt or solvate thereof or pharmaceutical compositions thereof in the manufacture of a medicament for the treatment of Periodic Limb Movement Disorder (PLMD).

A further aspect of the invention provides a method of treating Periodic Limb Movement Disorder (PLMD) in a mammal (human or animal subject) comprising the administration to said subject of an effective amount of (+)-(2S,3S)-2-(3-chlorophenyl)-3,5,5-trimethyl-2-morpholinol or a salt or solvate thereof or pharmaceutical compositions thereof.

One further aspect of the present invention provides the use of enantiomerically pure (+)-(2S,3S-2-(3-chlorophenyl)-3,5,5-trimethyl-2-morpholinol or a salt or solvate thereof or pharmaceutical compositions thereof in the manufacture of a medicament for the treatment of Periodic Limb Movement Disorder (PLMD).

A yet further aspect of the invention provides a method of treating Periodic Limb Movement Disorder (PLMD) in a mammal (human or animal subject) comprising the administration to said subject of an effective amount of enantiomerically pure (+)-(2S,3S-2-(3-chlorophenyl)-3,5,5-trimethyl-2-morpholinol or a salt or solvate thereof or pharmaceutical compositions thereof.

### DETAILED DESCRIPTION OF THE INVENTION

It will be appreciated that references herein to “treatment” extend to prophylaxis, prevention of recurrence and suppression or amelioration of symptoms (whether mild, moderate or severe) as well as the treatment of established conditions.

As used herein, “Restless Legs Syndrome (RLS)” is also known as the Ekbom syndrome and is a sensorimotor disorder with a general-population prevalence of 5-10%. RLS is an internationally recognised disorder listed in the diagnostic manuals ICD 10 (Chapter VI, G25.8; World Health Organisation, Geneva, 1994), DSM IV (Dyssomnia not otherwise specified 307.47) and also in the American Sleep Association International Classification of Sleep Disorders (ICSD) (Thorpy M. J, Chairman ICSD, Diagnostic Classification Steering Committee, Rochester Minn., 1990). RLS is characterised by stereotypical jerks of the lower limbs, typically during sleep (including periodic limb movements (PLMs)). RLS may also be characterised as an unpleasant twitching, burning or painful sensation, likened by sufferers to ‘crawling ants’ or ‘writhing worms’ in the muscles and bones which usually occurs during the evenings. The sensations usually occur in the calf, sometimes in the thighs and feet and once they have begun, there is an irresistible urge to move the legs to release the feelings and general discomfort. Symptoms are worse or exclusively present at rest, in the evenings and at night, and are relieved by movement. The need to move occurs on average every 20 to 40 seconds and the movements last for about 1 to 5 seconds. For some patients, RLS is mild and causes little inconvenience, in others however, the impact on sleep is considerable, compromising work and social activities (Allen, R. P. and Earley, C. J. (2001) J Clin Neurophysiol;18:128-147 and Earley, C. J. (2003) N Engl J Med; 348:2103-2109). A minority of RLS cases are secondary to a pre-existing condition (pregnancy, renal failure and iron-deficiency anaemia), and resolve with that underlying condition. Minimal criteria for the diagnosis of RLS were published by the International Restless Legs Syndrome Study Group (IRLSSG) in 1995 (Walters, A. S. (1995) Mov Disord;10:634-642) and updated in 2003 (Allen et al. (2003) Sleep Med;4:101-119).

The use of the compound of formula (I) or a salt or solvate thereof in the treatment of RLS may result in improvement in the subject's condition as determined by one or more of the following clinical measures, following administration: PLMI (periodic limb movement index), PLMAI (periodic leg movement with arousal index), PLMW (periodic leg movements during wakefulness), and IRLS (International Restless Leg Syndrome) rating scale, although other measures may also be used as appropriate (for example Clinical Global Improvement score, domains of Medical Outcomes Study (MOS) Sleep Scale, St. Mary's Sleep Questionnaire Scale, and other measures of discomfort, sleep efficiency, sleep latency, or % sleep time in various stages in the sleep cycle).

In addition, the compound of formula (I) may be less prone to cause augmentation in the treatment of RLS than is the case with traditional agents such as L-Dopa.

Periodic Limb Movement Disorder (PLMD) is a condition related to RLS, also referenced in the American Sleep Association Intemational Classification of Sleep Disorders (ICSD) (Thorpy M. J, Chairman ICSD, Diagnostic Classification Steering Committee, Rochester Minn., 1990). PLMD is characterized by repetitive stereotyped movements of the limbs during sleep. The movements, while most common in the legs, can also affect the arms. The sufferer may or may not notice the movements while sleeping. These movements typically occur every 20 to 40 seconds, and may be associated with repeated arousal, and severely fragmented sleep. Generally, to be diagnosed with the disorder, the periodic limb movements (PLMs) will occur five or more times during each hour of sleep. The PLMs are most common in the stage of sleep known as non-REM (Rapid Eye Movement) sleep, which usually occurs during the first half of the night. The disorder may cause poor sleep and/or subsequent daytime somnolence.

Although RLS and PLMD both affect the limbs—and both affect a person's ability to sleep at night and function normally during the day—they are two different disorders. The movements of RLS occur most often when a person is awake and are a voluntary response to uncomfortable or painful feelings in the legs. The movements of PLMD occur most often when a person is asleep and are involuntary (not consciously controlled). People with periodic limb movements are often not aware of these movements, although on rare occasions they may notice the involuntary movement of PLMD while they are still awake. It has been estimated that about 80% of RLS patients also have periodic limb movement disorder; however, patients with PLMD often do not have RLS.

The use of the compound of formula (I) or a salt or solvate thereof in the treatment of PLMD may result in improvement in the subject's condition as determined by one or more of the following clinical measures, following administration: PLMI (periodic limb movement index), and PLMAI (periodic leg movement with arousal index), although other measures may also be used as appropriate.

As used herein, “enantiomerically pure” means that the composition contains greater than about 90% of the desired enantiomer by weight, preferably greater than about 95% of the desired enantiomer by weight, more preferably greater than about 99% of the desired enantiomer by weight, most preferably greater than 99.5% of the desired enantiomer by weight, said weight percent based upon the total weight of the compound of formula (I).

Preferred for use according to the present invention are pharmaceutically acceptable salts or solvates of the compound of formula (I), particularly those disclosed in U.S. Pat. No. 6,342,496 B1, U.S. Pat. No. 6,337,328 B1, U.S. Pat. No. 6,391,875 B1, U.S. Pat. No. 6,274,579 B1, U.S. Patent Application Publication Nos. 2002/0052340 A1, 2002/0052341 A1, and 2003/0027827 A1, as well as WO 01/62257, WO 99/37305, WO 00/51546 and WO 01/62257. Suitable pharmaceutically acceptable salts can include, but are not limited to, hydrochloride salt, hydrogen sulfate salt and other sulfate salts, hydrogen phosphate salt and other phosphate salts, methanesulfonate salt, p-toluenesulfonate salt, citrate salt, fumarate salt, tartrate salt, and the like. Of these, (+)-(2S, 3S)2-(3-chlorophenyl)-3,5,5-trimethyl-2-morpholinol hydrochloride is particularly preferred.

### Preparation

The compound of formula (I) or a salt or solvate thereof may be prepared in isolated form, and preferably in an enantomerically pure form, in accordance with the procedures set forth in WO 99/37305, US2003-0064988, US2003-0032643 and US2003-0027827 (all of Glaxo Group Limited) or WO 00/51546 and WO 01/62257 (both of Sepracor Inc.) the procedures of which are herein incorporated by reference.

### Dosage and Formulation

The compound of formula (I) or a salt or solvate thereof is administered in isolated form, and is preferably administered in an enantiomerically pure form.

The amount of compound of formula (I) or a salt or solvate thereof required to achieve the desired therapeutic effect will, of course depend on a number of factors, for example, the mode of administration and the recipient being treated. In general, the daily dose will be in the range of 0.02 to 5.0 mg/kg, more particularly 0.1 to 1.5 mg/kg, or 0.15 to 1.2 mg/kg. More particular ranges include 0.02 to 2.5 mg/kg, 0.02 to 1.0 mg/kg, 0.1 to 1.5 mg/kg, 0.02 to 0.25 mg/kg, 0.02 to 0.15 mg/kg and 0.02 to 0.07 mg/kg given as a single once a day dose or as single or divided doses throughout the day. Preferably in the treatment of RLS, administration will be at appropriate time(s) of the day so that a peak of plasma concentration of the compound of formula (I) coincides with late evening or bedtime.

The compound of formula (I) or a salt or solvate thereof may be employed in the treatment of Restless Legs Syndrome (RLS) as the compound per se, but is preferably presented with one or more pharmaceutically acceptable carriers, diluents or excipients in the form of a pharmaceutical formulation. The carriers, diluents and exipients must, of course, be acceptable in the sense of being compatible with the other ingredients of the formulation and must not be deleterious to the recipient. The carrier may be a solid or a liquid, or both, and is preferably formulated with the agent as a unit-dose formulation, for example, a tablet containing 1 mg, 2 mg, 5 mg, 10 mg, 20 mg, 40 mg, 60 mg, 80 mg, 100 mg, 120 mg, 150 mg and 200 mg of the compound of formula (I) or a salt or solvate thereof, more preferably 10-80 mg of the compound of formula (I) or a salt or solvate thereof. Suitable formulations for use in the present invention include sustained release solid-dosage formulations, optionally film-coated solid-dosage formulations, and especially tablet and caplet formulations, for oral administration of the compound of formula (I), particularly once-daily administration, for example those illustrated in Examples 1 to 5 below.

The formulations include those suitable for oral, rectal, topical, buccal (e.g. sub-lingual) and parenteral (e.g. subcutaneous, intramuscular, intradermal or intravenous) administration.

Formulations suitable for buccal (sub-lingual) administration include lozenges comprising a compound of formula (I) or a salt or solvate thereof in a flavoured base, usually sucrose and acacia or tragacanth, and pastilles comprising the agent in an inert base such as gelatin and glycerin or sucrose and acacia.

Formulations of the present invention suitable for parenteral administration conveniently comprise sterile aqueous preparations of a compound of formula (I) or a salt or solvate thereof, preferably isotonic with the blood of the intended recipient. These preparations are preferably administered intravenously, although administration may also be effected by means of subcutaneous, intramuscular, or intradermal injecton. Such preparations may conveniently be prepared by admixing the agent with water and rendering the resulting solution sterile and isotonic with the blood.

Formulations suitable for rectal administration are preferably presented as unit-dose suppositories. These may be prepared by admixing a compound of formula (I) with one or more conventional solid carriers, for example, cocoa butter, and then shaping the resulting mixture.

Formulations suitable for topical application to the skin preferably take the form of an ointment, cream, lotion, paste, gel, spray, transdermal patch, aerosol, or oil. Carriers which may be used include vaseline, lanolin, polyethylene glycols, alcohols, and combinations of two or more thereof.

It should be understood that in addition to the ingredients particularly mentioned above, the formulations may include other agents conventional in the art having regard to the type of formulation in question.

### FORMULATION EXAMPLES

The following non-limiting examples illustrate suitable formulations for use in the present invention, particularly for once-daily administration.

### Example 1

| | Amount/ |
|---|---|
| Component | unit (mg) |
| Tablet Core | |
| Formula (I)•HCl | 22.86(*) |
| Microcrystalline Cellulose (Avicel PH102) Ph. Eur/USNF | 176.20 |
| Hydroxypropylmethylcellulose (Methocel E4M CR) | 120.25 |
| Ph. Eur/USNF | |
| Sodium Bisulphate | 3.25 |
| Purified Water (removed during processing) Ph. Eur | qs |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 20 mg of the compound of formula (I)

### Example 2

| | Amount/ |
|---|---|
| Component | unit (mg) |
| Tablet Core | |
| Formula (I)•HCl | 45.72(*) |
| Microcrystalline Cellulose (Avicel PH102) Ph. Eur/USNF | 159.84 |
| Hydroxypropylmethylcellulose (Methocel E4M CR) | 113.75 |
| Ph. Eur/USNF | |
| Sodium Bisulphate | 3.25 |
| Purified Water (removed during processing) Ph. Eur | qs |
| Magnesium Stearate Ph. Eur/USNF | 2.44 |
| TOTAL | 325.00 |
| Tablet coating | 13.00 |
| Opadry White: OY-S-28876 | |
| TOTAL | 338.00 |

(*)corresponding to 40 mg of the compound of formula (I)

### Example 3

| | Amount/ |
|---|---|
| Component | unit (mg) |
| Tablet Core | |
| Formula (I)•HCl | 68.58(*) |
| Microcrystalline Cellulose (Avicel PH102) Ph. Eur/USNF | 153.23 |
| Hydroxypropylmethylcellulose (Methocel E4M CR) | 97.50 |
| Ph. Eur/USNF | |
| Sodium Bisulphate | 3.25 |
| Purified Water (removed during processing) Ph. Eur | qs |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 60 mg of the compound of formula (I)

### Example 4

| | Amount/ |
|---|---|
| Component | unit (mg) |
| Tablet Core | |
| Formula (I)•HCl | 91.44(*) |
| Microcrystalline Cellulose (Avicel PH102) Ph. Eur/USNF | 130.37 |
| Hydroxypropylmethylcellulose (Methocel E4M CR) | 97.50 |
| Ph. Eur/USNF | |
| Sodium Bisulphate | 3.25 |
| Purified Water (removed during processing) Ph. Eur | qs |
| Magnesium Stearate Ph. Eur/USNF | 2.44 |
| TOTAL | 325.00 |
| Tablet coating | |
| Opadry White: OY-S-28876 | 13.00 |
| TOTAL | 338.00 |

(*)corresponding to 80 mg of the compound of formula (I)

### Example 5

| | Amount/ |
|---|---|
| Component | unit (mg) |
| Tablet Core | |
| Formula (I)•HCl | 11.43(*) |
| Microcrystalline Cellulose (Avicel PH102) Ph. Eur/USNF | 160.87 |
| Hydroxypropylmethylcellulose (Methocel E4M CR) | 146.25 |
| Ph. Eur/USNF | |
| Sodium Bisulphate | 3.25 |
| Purified Water (removed during processing) Ph. Eur | qs |
| Magnesium Stearate Ph. Eur/USNF | 3.25 |
| TOTAL | 325.05 |
| Tablet coating | |
| Opadry White: YS-1R-7003 | 9.75 |
| TOTAL | 334.80 |

(*)corresponding to 10 mg of the compound of formula (I)

Examples 1 to 5 above were prepared by a process similar to the following general process: The drug substance is blended and wet granulated with the pharmaceutically acceptable excipients described, including HPMC as the rate-controlling polymer. The acidic stabiliser (sodium bisulphate) is first dissolved in purified water to produce the granulation solution, and the granule is then produced by conventional processing techniques, for example either high shear or a fluid bed process, followed by drying, milling, blending, compression into a tablet, and finally aqueous film-coating.

### Biological Data

In Vitro Synaptosomal Uptake

In vitro uptake was determined, as reported previously, using synaptosomes prepared from rat caudoputamen (for dopamine uptake) and hypothalamus (for NA and serotonin uptake) using [³H]-dopamine, [³H]-NA and [³H]-serotonin as transport substrates, respectively. See Eckhardt, S. B., R. A. Maxwell, and R. M. Ferris, A Structure-Activity Study of the Transport Sites for the Hypothalamic and Striatal Catecholamine Uptake Systems. Similarities and differences. Molecular Pharmacology, 21: p. 374-9,1982

Synaptosomes for use in obtaining in vitro uptake data were prepared from hypothalamus or striatum by gently homogenizing the tissue in a 0.3 M sucrose/25 mM Tris pH 7.4 buffer containing iproniazid phosphate to inhibit monoamine oxidase. The homogenate was centrifuged at 1100×g at 4° C. for 10 min and the supernatant was used for uptake studies. The supernatant (˜1 mg tissue protein) was incubated with Km concentrations of [³H]-noradrenaline, [³H]-dopamine or [³H]-serotonin at 37° C. for 5 minutes in Modified Krebs-Henseleit buffer (118 mM NaCl, 5 mM KCl, 25 mM NaHCO₃, 1.2 mM NaH₂PO₄, 1.2 mM MgSO₄, 11 mM Dextrose, 2.5 mM CaCl₂) in the absence and presence of drug. Under these conditions uptake was linear with respect to both for substrate and tissue (with <5% total substrate transported). Non-specific uptake was defined as uptake at 0° C. [³H]-substrate, which had been transported into synaptosomes, was separated from free [³H]-substrate by filtration over GF/B filters and washing with cold Krebs-Henseleit buffer. The filters were counted for tritum in a liquid scintillation spectrometer.

The data for in vitro synaptosomal uptake are presented below as Table 1. The compound of formula (I), inhibited noradrenaline (NA) uptake with an IC₅₀ of 1.1 μM. On dopamine (DA) uptake, the compound of formula (I) had an IC₅₀ of ˜10 μM. The compound of formula (I) showed no inhibition of serotonin uptake at 30 μM.

TABLE 1

| Compound | IC₅₀ NA | IC₅₀ DA | IC₅₀ Serotonin |
|---|---|---|---|
| Formula (I) | 1.1 ± 0.07 | 9.3 ± 0.41 | >30 |

Uptake values are means i SEM of 3 separate experiments. The IC₅₀ values are concentrations (EM) required for 50% inhibition of uptake.
   Functional Reuptake Inhibition on Human Monoamine Transporters

Three separate cell-lines expressing human monoamine transporters for dopamine (hDAT) noradrenaline (hNET) and serotonin (hSERT) were used to measure the functional reuptake inhibiting properties of the compound of formula (I) (as its hydrochloride salt). The following methods were utilised.

Human noradrenaline transporter (hNET): MDCK/hNET (dog kidney) cells (4×10⁴ cells/well) expressing the human norepinephrine transporter were plated on 96-well format one day before the assay. When the cells were 80% confluent, cell monolayers were washed and preincubated with test compound and/or vehicle in modified Tris-HEPES buffer pH 7.1 at 25° C. for 20 minutes, then 25 nM [³H]Norepinephrine was added to make the total volume to 200 μl and the cells were further incubated for 10 minutes. Cells in the well were then rinsed twice, solubilized with 1% SDS lysis buffer and the lysate was counted to determine [³H]Norepinephrine uptake. Non-specific signal was determined in the presence of 10 μM desipramine. Reduction of [³H]Norepinephrine uptake by 50 per cent or more (≧50%) relative to vehicle controls indicated significant inhibitory activity.

Human dopamine transporter (hDAT): CHO-K1/hDAT cells (8×10⁴ cells/well) expressing the human dopamine transporter (hDAT) were plated on 96-well format one day before the assay. Cells were preincubated with test compound and/or vehicle in modified Tris-HEPES buffer pH 7.1 at 25° C. for 20 minutes, then 50 nM [³H]Dopamine was added to make the total volume to 200 μl and further incubated for 10 minutes. Cells in the well were then rinsed twice, solubilized with 1% SDS lysis buffer and the lysate was counted to determine [³H]Dopamine uptake. Non-specific signal was determined in the presence of 10 μM nomifensine. Reduction of [³H]Dopamine uptake by 50 per cent or more (≧50%) relative to vehicle controls indicates significant inhibitory activity.

Human serotonin transporter (hSERT): HEK-293/hSERT cells (5×10⁴ cells/tube) expressing the human serotonin transporter (hSERT) were added into the minitube on 96-tube holder prior to assay. Cells were preincubated with test compound or vehicle in modified Tris-HEPES buffer pH 7.1 at 25° C. for 20 minutes, then 65 nM [³H]Serotonin was added to make the total volume to 200 μl and further incubated for 10 minutes. Cells were then washed by filtration through cell harvester four times with PBS buffer containing 0.1% BSA and the GF/B filter was counted to determine [³H]Serotonin uptake. Non-specific signal was determined in the presence of 10 μM fluoxetine. Reduction of [³H]Serotonin uptake by 50 percent or more (≧50%) relative to vehicle-control indicates significant inhibitory activity.

Compounds were screened at 10, 1, 0.1, 0.01 and 0.001 μM. These same concentrations were concurrently applied to a separate group of untreated cells and evaluated for possible compound-induced cytotoxicity only if significant inhibition of uptake was observed. Radioactivity retained on the filters was determined by scintillation counting overnight using a Packard scintillation counter.

The potencies for monoamine reuptake inhibition for the hydrochloride salt of the compound of formula (I) are expressed in Table 2 below as IC₅₀ (in μM; mean±SEM) following three separate experiments, each performed in duplicate (n=3). The compound demonstrated reuptake inhibition at both hDAT (plC50=6.36) and hNET (plC50=6.70) but reuptake inhibition was not observed on hSERT (plC50<5) at the highest concentration tested (10 μM). No cytotoxicity was observed at any of the concentrations causing reuptake inhibition.

TABLE 2

| Compound | hNET | hDAT | hSERT |
|---|---|---|---|
| Formula (I).HCl | 0.20 ± 0.05 | 0.44 ± 0.01 | >10 |
| | (n = 3) | (n = 3) | (n = 3) |

Dopamine Transporter (DAT) Receptor Occupancy Study

The study was conducted in two parts. Part A utilized positron emission tomography (PET) to characterize the concentration-percent occupancy relationship with respect to the dopamine transporter (DAT) after dosing with intravenous compound of formula (I) to pseudo-steady-state. Part B utilized PET to evaluate the time course of occupancy at the dopamine transporter following steady-state dosing of the compound of formula (I) via a modified release oral formulation. The tracer used in this study to assess the occupancy at the DAT was ¹¹C-βCIT-FE.

Part A. Six healthy male volunteers each received a 20.6 mg intravenous dose of the compound of formula (I) administered via a 2-hour loading infusion followed by a 2-hour maintenance infusion to achieve pseudo-steady-state. Each subject then received two additional doses (61.8 mg and 91.2 mg) of the compound of formula (I), with each dose separated by 3 weeks. A baseline PET scan was performed between 1 and 7 days prior to first dosing with formula (I), and then each post-dose PET scan performed 2.5 hours after the initiation of each of the three dosing regimens.

The concentrate solution for infusion was formulated to contain 10 mg free base equivalent per mL as the hydrochloride salt of the compound of formula (I), and consisted of a clear, colorless solution, pH adjusted to approximately 4.5, buffered with 50 mmol citrate buffer. The product was diluted to a total volume of 250 mL with 0.9% weight-to-volume ratio (w/v) sodium chloride injection prior to being administered.

The study showed that the compound of formula (I) bound effectively to dopamine transporter in the striatum. A clear concentration-dependent transporter occupancy was observed, with receptor occupancy values of 14±0.38%, 37±8.0% and 47±6.8% being observed for the 20.6 mg, 61.8 mg, and 91.2 mg doses respectively.

Part B. Six healthy male volunteers received 60 mg of the compound of formula (I) via a modified release oral formulation once daily for 6 days in order to achieve steady-state by the last day. Four ¹¹C-βCIT-FE PET scans were performed for each subject, the first between 1 and 7 days prior to dosing and the remaining three at 6, 12 and 24 hours following the final dose on Day 6. The oral formulations were uncoated tablets, containing 20 mg and 40 mg of the compound of formula (I) as the hydrochloride salt details of the formulations are set out below as Examples 6 and 7 (prepared by an analogous method to Examples 1 to 5 above).

This oral dosing of 60 mg of the compound of formula (I) resulted in an average receptor occupancy of 29% (29±5.3%) at 6 hours after the last dose of the drug. Further PET investigations showed that the degree of receptor occupancy remained at almost the same level, on average 27% (27±11.3%), during the following 6 hours. The results from Part B of the study were in accordance with those from Part A.

| | | Amount/ |
|---|---|---|
| Example | Components | unit (mg) |
| 6 | Formula (I)•HCl | 45.72(*) |
| | Microcrystalline Cellulose | 70.30 |
| | (Avicel PH-102) EP/USP | |
| | Hydroxypropyl Methylcellulose | 81.26 |
| | (Methocel E4M CR) EP/USP | |
| | Sulfuric Acid EP/USP | 2.72 |
| | Purified Water (removed during processing) | qs |
| | EP/USP | |
| | Lactose monohydrate spray dried EP/USP | 121.75 |
| | Magnesium stearate EP/USP | 3.25 |
| | TOTAL | 325.00 |
| 7 | Formula (I)•HCl | 22.86(^{§}) |
| | Microcrystalline Cellulose | 93.16 |
| | (Avicel PH-102) EP/USP | |
| | Hydroxypropyl Methylcellulose | 81.26 |
| | (Methocel E4M CR) EP/USP | |
| | Sulfuric Acid EP/USP | 2.72 |
| | Purified Water (removed during processing) | qs |
| | EP/USP | |
| | Lactose monohydrate spray dried EP/USP | 121.75 |
| | Magnesium stearate EP/USP | 3.25 |
| | TOTAL | 325.00 |

(*)corresponding to 40.00 mg of the compound of formula (I)
(^{§})corresponding to 20.00 mg of the compound of formula (I)

The biological data presented above demonstrates that the compound of formula (I) inhibits the dopamine transporter in human subjects. Thus the compound of formula (I) may, as a result of enhancing dopaminergic activity in patients suffering from RLS or from PLMD, be an effective treatment for RLS or for PLMD.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. (canceled)

2. (canceled)

3. (canceled)

4. A method of treating Restless Legs Syndrome (RLS) in a mammal comprising the administration to said subject of an effective amount of (+)-(2S,3S)-2-(3-chlorophenyl)-3,5,5-trimethyl-2-morpholinol or a salt or solvate thereof or pharmaceutical compositions thereof.

5. The method according to claim 4 wherein said (+)-(2S,3S)-2-(3-chlorophenyl)-3,5,5-trimethyl-2-morpholinol or a salt or solvate thereof is enantiomerically pure.

6. The method according to claim 4 wherein the salt is (+)-(2S,3S)-2-(3-chlorophenyl)-3,5,5-trimethyl-2-morpholinol hydrochloride.

7. (canceled)

8. (canceled)

9. (canceled)

10. A method of treating Periodic Limb Movement Disorder in a mammal comprising the administration to said subject of an effective amount of (+)-(2S,3S)-2-(3-chlorophenyl)-3,5,5-trimethyl-2-morpholinol or a salt or solvate thereof or pharmaceutical compositions thereof.

11. The method according to claim 10 wherein said (+)-(2S,3S)-2-(3-chlorophenyl)-3,5,5-trimethyl-2-morpholinol or a salt or solvate thereof is enantiomerically pure.

12. The method according to claim 10 wherein the salt is (+)-(2S,3S)-2-(3-chlorophenyl)-3,5,5-trimethyl-2-morpholinol hydrochloride.

13. The method according to claim 4 wherein said mammal is a human.

14. The method according to claim 10 wherein said mammal is a human.
